(19) United States
(12) Patent Application Publication
(10) Pub. No.: US 2022/0072013 A1
(43) Pub. Date: Mar. 10, 2022

(54) Pharmaceutical Combinations for the Treatment of Cancer

(71) Applicant: CA*TX, Inc., Gladwyne, PA (US)

(72) Inventor: Bruce M. BOMAN, Gladwyne, PA (US)

(21) Appl. No.: 17/416,648

(22) PCT Filed: Dec. 20, 2019
(86) PCT No.: PCT/US2019/068029

§ 371 Date: Jun. 21, 2021

Related U.S. Application Data
(60) Provisional application No. 62/783,911, filed on Dec. 21, 2018.

(51) Int. Cl. A61K 31/58; A61K 31/343; A61K 38/19; A61P 35/00; A61K 45/06; A61K 31/192; A61K 31/616

(52) U.S. Cl. CPC A61K 31/58; A61K 31/343; A61K 38/19; A61K 31/616; A61K 45/06; A61K 31/192; A61P 35/00

(57) ABSTRACT

The disclosure provides combination therapies for the treatment of colorectal cancer and familial adenomatous polyposis (TAP).

### CROSS-REFERENCE TO RELATED APPLICATIONS

This application is an international application claiming the benefit of U.S. Provisional Application No. 62/783,911, filed on Dec. 21, 2018, which is incorporated herein by reference in its entirety.

### FIELD OF THE INVENTION

The invention relates generally to the field of medicine, cancer biology and treatment, and in particular to novel drug and drug combination methods and therapeutic strategies for treating cancer, and pharmaceutical compositions comprising such combinations, and methods of making and use thereof.

### BACKGROUND

Numerous cancer-related therapeutics are under phase I or phase II clinical trial and evaluations at any particular time; however, most of them will fail to advance. In fact, it is estimated that more than 90% of cancer-related therapeutics will fail phase I or II clinical trial evaluation. The failure rate in phase III trials is almost 50%, and the cost of new drug development from discovery through phase III trials is between $0.8 billion and $1.7 billion and can take between eight and ten years. In addition, many patients fail to respond even to standard drugs that have been shown to be efficacious. For reasons that are not currently well understood or easily evaluated, individual patients may not respond to standard drug therapy. In some cases, administration of drug combinations may be more efficacious for treating cancer than drugs administered individually. These drug combinations may act synergistically to enhance the anti-cancer activity of the drugs. In some cases, drugs that are not particularly efficacious may find new and unexpected uses when combined with additional drug therapies.

Colorectal cancer (CRC) is the third most commonly diagnosed cancer and third leading cause of cancer-related mortality in the United States, with an estimated 141,000 cases of colon and rectal cancer diagnosed in 2011. Surgical excision of early noninvasive adenomas is curative, however, there are few effective treatment options for patients suffering from advanced forms of CRC, and the prognosis is often poor. Despite a prolonged latency phase, only few lesions are identified at a stage where they can be surgically excised. Mutations in the human APC tumor suppressor gene are linked to Familial Adenomatous Polyposis (FAP), an inherited cancer-prone condition in which numerous polyps are formed in the epithelium of the large intestine. The development of CRC is initiated by the aberrant outgrowth of adenomatous polyps from the colonic epithelium that ultimately evolve into aggressive carcinomas. About 85% of sporadic colorectal cancers have been reported to harbor APC truncating mutations.

### SUMMARY OF THE INVENTION

In one aspect, the disclosure provides a method of treating cancer, including administering to a subject in need thereof a therapeutically effective amount of budesonide, or a pharmaceutically acceptable salt, solvate, hydrate, cocrystal, or prodrug thereof. In some embodiments, the cancer is selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the cancer is selected from colon cancer, stomach cancer, breast cancer, lung cancer, prostate cancer, pancreatic cancer, melanoma, and urothelial cancer. In some embodiments, the cancer is colorectal cancer.

In one aspect, the disclosure provides a method of treating a condition or disorder in a subject having an APC mutation, including administering to the subject in need thereof a therapeutically effective amount of budesonide, or a pharmaceutically acceptable salt, solvate, hydrate, cocrystal, or prodrug thereof. In some embodiments, the condition or disorder is cancer. In some embodiments, the cancer is selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the cancer is selected from colon cancer, stomach cancer, breast cancer, lung cancer, prostate cancer, pancreatic cancer, melanoma, and urothelial cancer. In some embodiments, the cancer is colorectal cancer. In some embodiments, the method further includes diagnosing the APC mutation in the subject.

In another aspect, the disclosure provides a method of treating familial adenomatous polyposis (FAP), including administering to a subject in need thereof a therapeutically effective amount of budesonide, or a pharmaceutically acceptable salt, solvate, hydrate, cocrystal, or prodrug thereof. In some embodiments, the subject has an APC mutation.

In another aspect, the disclosure provides a method of treating cancer, including administering to a subject in need thereof a therapeutically effective amount of budesonide, or a pharmaceutically acceptable salt, solvate, hydrate, cocrystal, or prodrug thereof, and a therapeutically effective amount of an additional therapeutic agent, or a pharmaceutically acceptable salt, solvate, hydrate, cocrystal, or prodrug thereof. In some embodiments, the therapeutically effective amount of budesonide and the therapeutically effective amount of additional therapeutic agent are synergistic amounts. In some embodiments, the cancer is selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the cancer is selected from colon cancer, stomach cancer, breast cancer, lung cancer, prostate cancer, pancreatic cancer, melanoma, and urothelial cancer. In some embodiments, the cancer is colorectal cancer. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day.

In another aspect, the disclosure provides a method of treating a condition or disorder in a subject having an APC mutation, including administering to the subject in need thereof a therapeutically effective amount of budesonide, or a pharmaceutically acceptable salt, solvate, hydrate, cocrystal, or prodrug thereof, and a therapeutically effective amount of an additional therapeutic agent, or a pharmaceutically acceptable salt, solvate, hydrate, cocrystal, or prodrug thereof. In some embodiments, the therapeutically effective amount of budesonide and the therapeutically effective amount of additional therapeutic agent are synergistic. In some embodiments, the condition or disorder is a cancer selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the condition or disorder is a cancer selected from colon cancer, stomach cancer, breast cancer, lung cancer, prostate cancer, pancreatic cancer, melanoma, and urothelial cancer. In some embodiments, the condition or disorder is colorectal cancer. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day. In some embodiments, the method further includes diagnosing the APC mutation in the subject.

In another aspect, the disclosure provides a method of treating familial adenomatous polyposis (FAP), including administering to a subject in need thereof a therapeutically effective amount of budesonide, or a pharmaceutically acceptable salt, solvate, hydrate, cocrystal, or prodrug thereof, and a therapeutically effective amount of an additional therapeutic agent, or a pharmaceutically acceptable salt, solvate, hydrate, cocrystal, or prodrug thereof. In some embodiments, the therapeutically effective amount of budesonide and the therapeutically effective amount of additional therapeutic agent are synergistic. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day. In some embodiments, the subject has an APC mutation.

In another aspect, the disclosure provides a pharmaceutical composition including: a therapeutically effective amount of budesonide, or a pharmaceutically acceptable salt, solvate, hydrate, cocrystal, or prodrug thereof; an additional therapeutic agent, or a pharmaceutically acceptable salt, solvate, hydrate, cocrystal, or prodrug thereof; and a pharmaceutically acceptable excipient. In some embodiments, the therapeutically effective amount of budesonide and the therapeutically effective amount of additional therapeutic agent are synergistic. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein.

In another aspect, the disclosure provides a therapeutically effective amount of budesonide, or a pharmaceutically acceptable salt, solvate, hydrate, cocrystal, or prodrug thereof, for use in the treatment of cancer. In another aspect, the disclosure provides a therapeutically effective amount of budesonide, or a pharmaceutically acceptable salt, solvate, hydrate, cocrystal, or prodrug thereof, and therapeutically effective amount of an additional therapeutic agent, or a pharmaceutically acceptable salt, solvate, hydrate, cocrystal, or prodrug thereof, for use in the treatment of cancer. In some embodiments, the therapeutically effective amount of budesonide and the therapeutically effective amount of additional therapeutic agent are synergistic. In some embodiments, the cancer is selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the cancer is selected from colon cancer, stomach cancer, breast cancer, lung cancer, prostate cancer, pancreatic cancer, melanoma, and urothelial cancer. In some embodiments, the cancer is colorectal cancer. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day.

In another aspect, the disclosure provides a therapeutically effective amount of budesonide, or a pharmaceutically acceptable salt, solvate, hydrate, cocrystal, or prodrug thereof, for use in the treatment of a condition or disorder related to an APC mutation. In another aspect, the disclosure provides a therapeutically effective amount of budesonide, or a pharmaceutically acceptable salt, solvate, hydrate, cocrystal, or prodrug thereof, and therapeutically effective amount of an additional therapeutic agent, or a pharmaceutically acceptable salt, solvate, hydrate, cocrystal, or prodrug thereof, for use in the treatment of a condition or disorder related to an APC mutation. In some embodiments, the therapeutically effective amount of budesonide and the therapeutically effective amount of additional therapeutic agent are synergistic. In some embodiments, the condition or disorder is a cancer selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia. In some embodiments, the condition or disorder is a cancer selected from colon cancer, stomach cancer, breast cancer, lung cancer, prostate cancer, pancreatic cancer, melanoma, and urothelial cancer. In some embodiments, the condition or disorder is colorectal cancer. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day.

In another aspect, the disclosure provides a therapeutically effective amount of budesonide, or a pharmaceutically acceptable salt, solvate, hydrate, cocrystal, or prodrug thereof, for use in the treatment of adenomatous polyposis (FAP). In another aspect, the disclosure provides a therapeutically effective amount of budesonide, or a pharmaceutically acceptable salt, solvate, hydrate, cocrystal, or prodrug thereof, and therapeutically effective amount of an additional therapeutic agent, or a pharmaceutically acceptable salt, solvate, hydrate, cocrystal, or prodrug thereof, for use in the treatment of adenomatous polyposis (FAP). In some embodiments, the therapeutically effective amount of budesonide and the therapeutically effective amount of additional therapeutic agent are synergistic. In some embodiments, the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil. In some embodiments, the additional therapeutic agent is selected from bevacizumab, ramucirumab, and ziv-aflibercept. In some embodiments, the additional therapeutic agent is selected from cetuximab, panitumumab, erlotinib, trastuzumab, lapatinib, pertuzumab, and trastuzumab emtansine. In some embodiments, the additional therapeutic agent is selected from regorafenib, sorafenib, and apatinib. In some embodiments, the additional therapeutic agent is a check-point inhibitor of the PD-1 receptor or the CTLA-4 receptor. In some embodiments, the additional therapeutic agent is napabucasin. In some embodiments, the additional therapeutic agent is selected from sulindac, aspirin, celecoxib, difluoromethylornithine (DFMO), sodium butyrate, cyclosomatostatin, somatostatin, glucagon and antabuse. In some embodiments, the additional therapeutic agent is a glucagon-like peptide. In some embodiments, the additional therapeutic agent is a statin. In some embodiments, the additional therapeutic agent is an all-trans retinoid or a 9-cis retinoid. In some embodiments, the additional therapeutic agent is a survivin inhibitor. In some embodiments, the additional therapeutic agent is a TCF-4 inhibitor. In some embodiments, the additional therapeutic agent is a bone morphogenic protein. In some embodiments, budesonide and the additional therapeutic agent are administered concurrently. In some embodiments, budesonide and the additional therapeutic agent are administered sequentially within about 1 hour to about 24 hours of each other. In some embodiments, budesonide and the additional therapeutic agent are administered sequentially within about 1 day to about 7 days of each other. In some embodiments, budesonide and the additional therapeutic agent are co-formulated in a pharmaceutical composition. In some embodiments, budesonide and the additional therapeutic agent are administered daily, every other day, or every third day. In some embodiments, the FAP is APC mutated FAP.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 illustrates the decrease in the number of intestinal tumors in Apc^{Min/+} mice given Budesonide- or Sulindac-containing diets for 8 or 4 weeks (from age 4 weeks to age 12 weeks or age 8 weeks to age 12 weeks); bars show % change in tumor number compared to untreated C57BL/6 control mice. Each diet group had a total of 12 mice, six starting treatment at 4 weeks of age (before tumor initiation) and six starting at 8 weeks of age (after tumor initiation).

FIG. 2 illustrates the change in size of intestinal tumors in Apc^{Min/+} mice that were administered Budesonide- or Sulindac containing diets for 8 or 4 weeks (from age 4 weeks to age 12 weeks or age 8 weeks to age 12 weeks); bars show % change in tumor size compared to untreated C57BL/6 control mice. Tumor size decreased when Budesonide or Sulindac was started before, but not after adenoma development.

FIGS. 3A-3D illustrate that Sulindac attenuates expression of the anti-apoptotic protein survivin in human CRC cells; survivin and Bcl-2 expression was evaluated in HT-29 cells that were treated with Sulindac (200 μM). RNA and protein expression were measured by RT-PCR and western blot, respectively. FIGS. 3A and 3B show an ethidium bromide-stained gel and graph of quantified data for survivin and Bcl-2 mRNA expression. FIGS. 3C and 3D show a western blot and graph of quantified data for survivin and Bcl-2 protein expression.

FIGS. 4A-4D illustrate immunostaining using anti-5-methylcytidine antibodies to evaluate global DNA methylation; Budesonide increases global DNA methylation of intestinal tissues in Apc^{Min/+} Mice. Immunostaining signal intensity is decreased in the lower crypt region compared to the upper crypt and villous regions in normal-appearing small intestine of B6 controls (FIG. 4A) and Ap^{Min/+} mice (FIG. 4B). Immunostaining signal intensity is decreased in adenomas of the small intestine of Apc^{Min/+} mice (FIG. 4C). Budesonide treatment induces an increase in both the proportion and intensity of adenomatous cells staining positive for 5-methylcytidine (FIG. 4D).

FIG. 5 shows a circular ideogram of methylation load with hypermethylated genes (>2-fold increase compared to mean) designated in red text, and hypo-methylated genes (>2-fold decrease) in green text, illustrating that Budesonide increases DNA methylation in human CRC cells. Epigenetic profiling was done on HT29 (APC mutant) cells in response to Budesonide exposure, and the DNA methylation profiling revealed that an overall increase in methylation occurred across a large portion of the genome following Budesonide exposure (69.3% of 1 MB domains across the genome showed an increase in methylation in response to Budesonide versus control; a statistically significant shift in CpG methylation occurred at 235 unique CpG sites out of 1.9M that were analyzed).

### DETAILED DESCRIPTION

The disclosure provides a budesonide therapy for the treatment of cancer, in particular colorectal cancer, or FAP. The disclosure also provides combination therapies for the treatment of cancer, in particular colorectal cancer, or FAP, such as combination therapies of budesonide with other therapeutic agents, for example anticancer agents for treating cancer or FAP. Such combinations provide synergistic effects in the treatment of cancers or FAP, and particularly treatment of colorectal cancer.

### Definitions

The general terms used herein preferably have the following meanings within the context of this disclosure, unless otherwise indicated. Thus, the definitions of the general terms as used in the context of the present invention are provided herein below:

The singular forms “a,” “an,” and “the” include plural reference unless the context clearly dictates otherwise.

The term “about,” as used herein, generally refers to an acceptable error range for the particular value as determined by one of ordinary skill in the art, which may depend in part on how the value is measured or determined. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, and within 2-fold, of a value.

As used herein, the term “at least one” is refers to one or more. For instance, the term “at least one anticancer agent” means that the combination includes a single anticancer agent or more anticancer agents.

The term “effective amount” or “therapeutically effective amount,” as used herein, generally refers to an amount of a compound described herein that is sufficient to affect an intended, predetermined, or prescribed application, including but not limited to, disease or condition treatment. The therapeutically effective amount can vary depending upon the application (e.g., in vitro or in vivo), or the subject and disease condition being treated, e.g., the weight and age of the subject, the severity of the disease condition and the manner of administration. The term also may apply to a dose that induces a particular response in target cells, e.g., reduction of proliferation or down regulation of activity of a target protein. The specific dose may vary depending on the particular compounds chosen, the dosing regimen to be followed, whether it is administered in combination with other compounds, timing of administration, the tissue to which it is administered, and the physical delivery system in which it is carried.

As used herein, the term “pharmaceutically acceptable” means that the carrier, diluent, excipients, and/or salt must be compatible with the other ingredients of the formulation, and not deleterious to the recipient thereof “Pharmaceutically acceptable” also means that the compositions or dosage forms are within the scope of sound medical judgment, suitable for use for an animal or human without excessive toxicity, irritation, allergic response, or other problem or complication, commensurate with a reasonable benefit/risk ratio.

As used herein, the term “combination” or “pharmaceutical combination” refers to the combined administration of the anticancer agents. Combinations of the disclosure include budesonide and at least one therapeutic agent, including, but not limited to, an anticancer agent selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, tipiracil, bevacizumab, ramucirumab, ziv-aflibercept, cetuximab, panitumumab, erlotinib, trastuzumab, lapatinib, pertuzumab, trastuzumab emtansine, regorafenib, sorafenib, apatinib, a check-point inhibitor of the PD-1 receptor or the CTLA-4 receptor, napabucasin, sulindac, aspirin, celecoxib, difluoromethylornithine (DFMO), sodium butyrate, cyclosomatostatin, somatostatin, glucagon, antabuse, a glucagon-like peptide, a statin, an all-trans retinoid (e.g., all trans retinoic acid, or ATRA), a 9-cis retinoid (e.g., 9-cis retinoic acid), a survivin inhibitor, a TCF-4 inhibitor, or a bone morphogenic protein, which therapeutic agents or anticancer agents may be administered to a subject in need thereof, e.g., concurrently or sequentially. Other anticancer agents are described herein, or can be contemplated by one skilled in the art.

The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 175%, about 200%, about 225%, about 250%, about 275%, about 300%, about 325%, about 350%, about 375%, about 400%, about 425%, about 450%, about 475%, or 500%, or more, than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. As used herein, “synergistic amounts” refer to such doses. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

As used herein, the term “adenomatous polyposis coli gene” or “APC gene” or “APC” refers to a mammalian DNA sequence coding for an APC protein. An example of a human APC gene is located at 5q21-q22 on chromosome 5, GenBank: M74088.1. Synonyms for the human APC gene include: BTPS2, DP2, DP2.5, DP3, PPP1R46 and “protein phosphatase 1, regulatory subunit 46.” An example of a mouse APC gene is located at chromosome 18 Bl, MGI:88039. Synonyms for the mouse APC gene include: CC2, Min, mAPC, AI0147805, AU020952, and AW124434.

The term “Adenomatous polyposis coli protein” or “APC protein” or “APC” as used herein refers to a mammalian protein sequence of 2843 amino acids. An example of a human APC sequence is GenBank: AAA03586. An example of a mouse APC sequence is GenBank: AAB59632.

The term “APC truncation” or “APC truncation mutant” or “APC truncation mutation” refers to a truncated protein product resulting from a mutation occurring within the APC gene. An APC truncation can be, for example, but not limited to, a 1309 amino acid product or a 1450 amino acid product.

The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

As used herein, the terms “subject,” “individual,” and/or “patient” are used interchangeably to refer to a member of an animal species of mammalian origin, including humans.

The term “inhibiting” as used herein refers to reducing or modulating the chemical or biological activity of a substance or compound.

The terms “disease,” “disorder,” and/or “condition,” as used herein, refer to an impairment of health or a condition of abnormal functioning.

The term “drug” as used herein refers to a therapeutic agent or any substance used in the prevention, diagnosis, alleviation, treatment, or cure of disease.

The term “therapeutic agent” as used herein refers to a drug, molecule, nucleic acid, protein, metabolite, composition, or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein.

The term “reduce” or “reducing” as used herein refers to limit occurrence of a disorder in individuals at risk of developing the disorder.

As used herein, the terms “subject” or “individual” or “patient” are used interchangeably to refer to a member of an animal species of mammalian origin, including humans.

The term “symptom” as used herein refers to a phenomenon that arises from and accompanies a particular disease or disorder and serves as an indication of it.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The terms “mutants” and “variants” are used interchangeably herein to refer to nucleotide sequences with substantial identity to a reference nucleotide sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of techniques.

The term “pharmaceutical composition” as used herein refers to a preparation comprising a pharmaceutical product, drug, metabolite, or active ingredient.

As used herein the terms “treat,” “treating,” and/or “treatment” include abrogating, substantially inhibiting, slowing or reversing the progression of a disease, disorder, or condition, substantially ameliorating clinical symptoms of a disease, disorder, or condition, or substantially preventing the appearance of clinical symptoms of a disease, disorder, or condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); (e) limiting recurrence of symptoms in patients that were previously asymptomatic for the disorder(s); (f) alleviating, abating or ameliorating a disease or condition symptoms; (g) preventing additional symptoms, ameliorating or preventing the underlying causes of symptoms; (h) inhibiting the disease or condition; (i) arresting the development of the disease or condition; (j) relieving the disease or condition; (k) causing regression of the disease or condition; (l) relieving a condition caused by the disease or condition; and/or (m) stopping the symptoms of the disease or condition either prophylactically and/or therapeutically.

The term “condition” as used herein refers to a variety of health states and is meant to include disorders or diseases caused by any underlying mechanism or disorder in which a truncated APC protein is expressed. A subject in need thereof is a patient having, or at risk of having a disorder related to APC mutation.

As used herein, the term “therapeutic amount” and/or “therapeutically effective amount” refer to the amount of a therapeutic agent necessary or sufficient to realize a desired biologic effect. Combined with the teachings provided herein, by choosing among the various therapeutic agents and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular described therapeutic agent, the size of the subject, or the severity of the disease or condition. One of ordinary skill in the art may determine empirically the therapeutically effective amount of a particular described compound and/or other therapeutic agent without necessitating undue experimentation. It is generally preferred that a maximum dose be used, that is, the highest safe dose according to some medical judgment. The terms “dose” and “dosage” are used interchangeably herein. For any therapeutic agent described herein the therapeutically effective amount can be initially determined from preliminary in vitro studies and/or animal models. A therapeutically effective dose can also be determined from human data. The applied dose can be adjusted based on the relative bioavailability and potency of the administered compound. Adjusting the dose to achieve maximal efficacy based on the methods described above and other methods as are well-known in the art is within the capabilities of the ordinarily skilled artisan. Therapeutically effective amounts can be currently approved recommended dosages and amounts.

### DETAILED DESCRIPTION

The disclosure relates to the treatment of cancer, in particular colorectal cancer (CRC), and treatment of precancerous conditions such as familial adenomatous polyposis (FAP). Colorectal cancer and FAP are relatively common. CRC in particular is associated with high mortality rates. The disclosure provides methods for treating CRC and FAP by use of combination therapies including at least budesonide.

Conventional therapy for adult patients with advanced cancer consists of combinations of cytotoxic chemotherapeutic drugs (e.g. 5-FU based regimens for CRC) which usually are not curative and are associated with significant toxicity. Some newer “targeted agents” developed against altered molecular pathways in cancer are showing efficacy, but are not producing expected (>50%) tumor response rates and/or cancer cures. Immunotherapeutic agents (i.e. immune checkpoint inhibitors) offer a promising approach by boosting the body's natural defenses to fight cancer. However, immunotherapy is beneficial to only a small proportion (<10%) of all cancers, i.e. mainly to ones that have a high mutation rate. For CRC, only 3% of advanced stage tumors display a markedly elevated mutational rate (due to DNA mismatch repair or polymerase deficiency). Thus just a minority of patients will likely benefit from immunotherapy.

A majority of advanced CRCs (>80%) have inactivation of the tumor suppressor gene APC, and the APC mutation is the primary driver of tumor growth in most CRCs. APC, which does not act as a classical tumor suppressor, influences Wnt signaling thereby regulating gene transcription. Wnts are a family of secreted cysteine-rich glycoproteins that have been implicated in the regulation of stem cell maintenance, proliferation, and differentiation during embryonic development. Canonical Wnt signaling increases the stability of cytoplasmic 13-catenin by receptor-mediated inactivation of GSK-3 kinase activity and promotes 13-catenin translocation into the nucleus. The canonical Wnt signaling pathway also functions as a stem cell mitogen via the stabilization of intracellular 13-catenin and activation of the 13-catenin/TCF/LEF transcription complex, resulting in activated expression of cell cycle regulatory genes, such as Myc, cyclin Dl, EPhrinB (EPhB) and Msxl, which promote cell proliferation. APC is the negative regulator of Wnt signaling. Without this negative regulation, the Wnt pathway is more active and is important in cancer.

The APC gene product is a 312 kDa protein consisting of multiple domains, which bind to various proteins, including beta-catenin, axin, C-terminal binding protein (CtBP), APC-stimulated guanine nucleotide exchange factors (Asefs), Ras GTPase-activating-like protein (IQGAP1), end binding-I (EB1) and microtubules. APC suppresses canonical Wnt signaling, which is essential for tumorigenesis, development and homeostasis of a variety of cell types, including epithelial and lymphoid cells, but functions in several other fundamental cellular processes. These cellular processes include cell adhesion and migration, organization of actin and microtubule networks, spindle formation and chromosome segregation. Deregulation of these processes caused by mutations in APC is implicated in the initiation and expansion of colon cancer. The APC protein functions as a signaling hub or scaffold, in that it physically interacts with a number of proteins relevant to carcinogenesis. Loss of APC influences cell adhesion, cell migration, the cytoskeleton, and chromosome segregation, and it is believed that APC mutations cause a loss of function change in colon cancer. Missense mutations yield point mutations in APC, while truncation mutations cause the loss of large portions of the APC protein, including defined regulatory domains.

While loss of function due to APC may be partially correct, it is also believed that a large fraction of colon cancer patients have at least one APC gene product that is truncated, and that this has a gain of function. Thus truncated APC proteins may play an active role in colon cancer initiation and progression as opposed to being recessive; for example, truncated APC, but not full-length APC may activate Asef and promote cell migration. Although defects in APC occur in a high fraction of colon cancer cases, there are currently no therapeutics targeting vulnerabilities resulting from these defects.

Without wishing to be bound by any particular theory, it is believed that most advanced CRCs (>80%) have inactivation of the tumor suppressor gene APC. Also without wishing to be bound by any particular theory, it is believed that several lines of evidence show that APC mutations drive the development and growth of CRC: (i) APC mutations lead to constitutionally active WNT signaling via downstream activation of TCF4, which induces transcription of genes that drive cell proliferation, and WNT signalling is altered in ˜95% of human CRCs, primarily due to bi-allelic mutation of the APC gene; (ii) APC mutation is sufficient for early CRC development; familial adenomatous polyposis (FAP) patients, who have germline APC mutations, have 100% risk for developing CRC if left untreated; (iii) tumors that develop because of an APC mutation have a worse prognosis than tumors that develop because of DNA mismatch repair mutations; (iv) the severity of any given APC mutation (without wishing to be bound by any particular theory, it is believed that most lead to truncation of the APC protein and APC protein inactivation), correlates with the severity of the CRC; (v) the characteristics of the 2nd APC hit depend on the nature of the 1st APC hit; (vi) APC mutations are required for the maintenance of colon carcinomas; (vii) transfection of APC into CRC cells induces cell cycle arrest and apoptosis; (viii) restoring wild-type Apc in CRC leads to cellular differentiation and re-establishes crypt homeostasis; (ix) it has been shown that APC mutations that drive CRC growth do so by causing SC overpopulation.

The disclosure provides for the use of Apc^{Min} mice to screen for drugs that target APC. The only mutation carried by Apc^{Min} mice is a germline Apc mutation, and Apc^{Min} mice develop multiple intestinal tumors as a result. Budesonide, a steroidal anti-inflammatory drug active against lung cancer development, was investigated as a therapeutic agent against CRC, and was found to strongly decrease (by 88%) intestinal tumor development induced by Apc^{Min} mutation. Budesonide's effect was comparable to that of the non-steroidal anti-inflammatory drug (NSAID) Sulindac (90% decrease).

As described herein, Apc^{Min} mice can be used to screen drugs for their ability to target the mechanisms that are disrupted by APC mutation. The only mutation carried by these mice is a germline Apc mutation, and that Apc mutation by itself is sufficient to cause the mice to develop multiple intestinal tumors. Min mice have just the one tumor-initiating mutation (Apc^{Min}) and an acquired second Apc mutation leads to complete Apc inactivation and tumor development. Thus, any effect by a drug that inhibits the tumorigenic process is the result of the drug's ability to modify neoplastic tissue and offset the APC-based mechanisms that drive tumor growth. Without wishing to be bound by any particular theory, it is believed that a tactic to discover new drugs against malignant colonic tumors (human CRC) is by evaluating agents that inhibit tumor development in Apc^{Min} mice, because: 1) one class of agents known to prevent CRC is also active against metastatic CRC; namely, NSAIDs (e.g., Sulindac, Celecoxib, aspirin) prevent Apc^{Min} tumors and human CRCs, and metastatic CRC patients taking NSAIDs have significantly increased survival; 2) APC mutations occur early in CRC initiation, so they will be carried by all tumor cells during CRC development (including cancer stem cells), and APC mutation is the primary driver of tumor growth in most CRCs.

The disclosure provides for identifying drugs that act against a variety of APC mutations. Using the Apc^{Min} model which has mainly been used in the past to identify chemopreventive drugs is applied as an innovative approach to identify drugs active against CRCs that can carry a variety of APC mutations. In one embodiment, this approach is used to identify Budesonide as a CRC candidate drug as described herein low. Without wishing to be bound by any particular theory, it is believed that while Apc^{Min} mice carry a tumor-predisposing germline Apc mutation, the intestinal tumors that spontaneously develop and grow in vivo are due to further inactivation (second hit) of Apc. Also without wishing to be bound by any particular theory, it is believed that this model is useful because any drug that has activity against a variety of APC mutations is effective against the many processes and pathways disrupted by different APC mutations.

The wild-type APC gene normally encodes a large scaffold-type protein that has seven major functional domains which interact and control at least 22 different proteins and their associated pathways (WNT being just one of these pathways). Thus, depending on the location of any given APC mutation, it can affect different cellular processes that promote tumor development in different ways. In the development of GI tumors in vivo in Min mice, the acquired (somatic) mutations that inactivate Apc's second allele is variable and many could be scattered throughout Apc's gene sequence. Thus, each different tumor that develops theoretically can have a different second hit in Apc. The variable Apc genotypes of multiple intestinal tumors in Min mice thus provide a model that simulates the situation in human CRCs whereby APC mutations in different human CRCs are variable and widely distributed across APC's sequence. Given that the nature (type and location) of the APC mutation can differ in the tumors among individual cancer patients, it is important to identify drugs that are effective against any of the alterations in APC that can disrupt the function of the many cellular pathways that APC controls, and also the effect of these alterations on tissue dynamics that drive cancer growth. Thus, without wishing to be bound by any particular theory, it is believed that the approach described herein is different from approaches that develop agents that target components in just one pathway (e.g. WNT signaling) or use a drug screen that involves just one specific mutation in APC. Without wishing to be bound by any particular theory, it is believed that because budesonide is effective in inhibiting the development and growth of most intestinal tumors in Apc^{Min} mice, budesonide is efficacious against tumors having different APC genotypes in CRC patients.

Without wishing to be bound by any particular theory, the disclosure is based, at least in part, on budesonide's preferential inhibition of growth of malignant human colonic cells that carry an adenomatous polyposis coli (APC) mutation. In some embodiments, this effect is enhanced when budesonide is combined with a nonsteroidal anti-inflammatory drug (NSAID). Also without wishing to be bound by any particular theory, it is believed that both budesonide and NSAIDs are anti-inflammatory drugs, but they act through different molecular mechanisms.

In some embodiments, budesonide inhibits the growth of a variety of human CRC cell lines. In one embodiment, cells with mutant APC are two fold (or more) as sensitive as wildtype APC cells to growth inhibition by Budesonide. Increased differentiation and apoptosis occurs in parallel with growth inhibition. In some embodiments, an NSAID, e.g., sulindac or aspirin, potentiate the anti-tumor activity of Budesonide. In some embodiments, budesonide and NSAIDs have synergistic effects.

The disclosure provides for selecting a drug combination that is synergistic. To create a novel drug combination that is synergistic, and without wishing to be bound by any particular theory, it is believed that a choice of drugs can be based on i) anti-tumor activity in Apc^{Min} model, ii) their mechanisms of action. The mechanisms of action of drugs can affect efficacy when administered in combination. For example, when two drugs act through the same or similar mechanisms, they often display additive activity when combined. Additive activity is considered to be equal to the sum of the effect of the two agents given separately. In contrast, and without wishing to be bound by any particular theory, it is believed if two drugs act through different mechanisms, the combination of the drugs can produce synergistic activity. That is, the effect of two agents administered together is greater than the sum of their separate effect at the same doses. To identify drugs, when administered in combination, that might have synergistic activity against advanced CRCs, different agents were selected that act via different mechanisms. In one embodiment, two anti-inflammatory drugs that not only have strong activity against tumor development in ApcMin mice were selected, but they also have different molecular mechanisms of action. Thus, because Budesonide and NSAIDs have different mechanisms of action, it is believed that when administered in combination, they have synergistic activity.

As described herein, the disclosure provides combination therapy including budesonide and one or more additional therapeutic agent. In some embodiments, the combination therapy is for the treatment of various cancers, including, but not limited to, colorectal cancer. In some embodiments, the combination therapy is for the treatment FAP. In some embodiments, the combination therapy si for the treatment of any condition in a subject having an PC mutation. As used herein, combination therapy includes both methods of treatment and compositions of matter including one or more therapeutic agents.

In one embodiment, the therapeutic agent is budesonide:

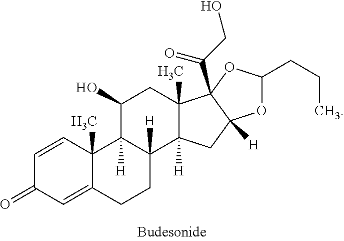

In some embodiments, budesonide is administered rectally (PR). In some embodiments, budesonide is administered as 1 metered dose (2 mg) PR BID for 2 weeks followed by 1 metered dose PR once daily in evening for 4 weeks.

In some embodiments, budesonide is administered orally (PO). In some embodiments, budesonide is administered as capsules, for example enteric-coated and/or extended-release capsules. In some embodiments, budesonide is administered as a 3 mg tablet, for example enteric-coated and/or extended-release tablet. In some embodiments, budesonide is administered as a 9 mg dose. In some embodiments, budesonide is administered in a 9 mg dose PO qAM for up to 8 weeks. In some embodiments, budesonide is administered in a 9 mg dose PO qAM for up to 8 weeks, and 8-week courses may be repeated. In some embodiments, budesonide is administered in a 6 mg dose PO qAM. In some embodiments, budesonide is administered in a 6 mg dose PO qAM for up to 3 months.

In some embodiments, budesonide can be administered by inhalation. In some embodiments, budesonide is administered as an aerosol for example at a dose of (80 mcg/4.5 mcg)/actuation, or at a dose of (160 mcg/4.5 mcg)/actuation. In some embodiments, budesonide is administered as an aerosol at a dose of 160 mcg/9 mcg (2 actuations of 80 mcg/4.5 mcg) q12 hr, or at a dose of 320 mcg/9 mcg (2 actuations of 160 mcg/4.5 mcg) q12 hr. In some embodiments, budesonide is administered as an aerosol at a dose of 160 mcg/9 mcg (2 actuations of 80 mcg/4.5 mcg) q12 hr, or 320 mcg/9 mcg q12 hr. In some embodiments, budesonide is administered as an aerosol at a dose of 160 mcg/4.5 mcg.

In some embodiments, budesonide can be administered intranasally. In some embodiments, budesonide is administered intranasally at a dose of 32 mcg/actuation. In some embodiments, budesonide is administered intranasally at a dose of 1 spray/nostril qDay (64 mcg/day), or at a dose of 4 sprays/nostril qDay (256 mcg/day). In some embodiments, budesonide is administered intranasally at a dose of 2 sprays/nostril qDay (128 mcg/day).

In some embodiments, the additional therapeutic agent is 5-fluorouracil. In some embodiments, 5-Fluorouracil (5-FU) is administered at a dose of 500 mg/m² IV on Days 1-5, or 450-600 mg/m² IV weekly, or 200-400 mg/m² IV continuous infusion qDay.

In some embodiments, the additional therapeutic agent is albumin-bound paclitaxel. In some embodiments, albumin-bound paclitaxel is administered at a dose of 125 mg/m² intravenously over 30-40 minutes on Days 1, 8, and 15 of each 28-day cycle. In some embodiments, albumin-bound paclitaxel is administered at a dose of 260 mg/m² IV infused over 30 minutes q3 weeks. In some embodiments, albumin-bound paclitaxel is administered at a dose of 100 mg/m² IV infused over 30 minutes on Days 1, 8, and 15 of each 21-day cycle.

In some embodiments, the additional therapeutic agent is capecitabine. In some embodiments, capecitabine is administered at a dose of 1250 mg/m² BID for 2 weeks q21 days, or 1000-1250 mg/m² PO BID daily on days 1-14, every 21 d.

In some embodiments, the additional therapeutic agent is carboplatin. In some embodiments, carboplatin is administered at a dose of 360 mg/m² IV q4 Weeks.

In some embodiments, the additional therapeutic agent is cisplatin. In some embodiments, cisplatin is administered at a dose of 50-100 mg/m² IV, for example on day 1. In some embodiments, cisplatin is administered at a dose of 20 mg/m²/day IV for 5 days/cycle. In some embodiments, cisplatin is administered at a dose of 50-70 mg/m² IV cycle q3-4 Weeks. In some embodiments, cisplatin is administered at a dose depending on prior radiation therapy or chemotherapy. In some embodiments, cisplatin is administered at a dose of 50 mg/m²/cycle. In some embodiments, cisplatin is administered at a dose repeated q4 Weeks. In some embodiments, cisplatin is administered at a dose of 75-100 mg/m² IV per cycle q4 Weeks. In some embodiments, cisplatin is administered at a dose of 90-270 mg/m² intraperitoneally. In some embodiments, cisplatin is administered at a dose repeated q3 Weeks. In some embodiments, cisplatin is administered at a dose of 75-100 mg/m² IV q4 Weeks. In some embodiments, cisplatin is administered at a dose of 100 mg/m² IV q4 Weeks.

In some embodiments, the additional therapeutic agent is dacarbazine. In some embodiments, dacarbazine is administered at a dose of 2-4.5 mg/kg IV qDay for 10 days. In some embodiments, dacarbazine is administered as a repeat dose q4 Weeks. In some embodiments, dacarbazine is administered at a dose of 250 mg/m² IV qDay for 5 days. In some embodiments, dacarbazine is administered as a repeat dose q3 Weeks. In some embodiments, dacarbazine is administered at a dose of 150 mg/m² IV qDay for 5 days, repeat q4 Weeks. In some embodiments, dacarbazine is administered at a dose of 375 mg/m² IV on Day 1; repeat every 15 Days.

In some embodiments, the additional therapeutic agent is docetaxel. In some embodiments, docetaxel is administered at a dose of 75-100 mg/m² IV on day 1. In some embodiments, docetaxel is administered every 21 d.

In some embodiments, the additional therapeutic agent is doxorubicin. In some embodiments, doxorubicin is administered at a dose of 60-75 mg/m² IV q21 Days. In some embodiments, doxorubicin is administered at a dose of 60 mg/m² IV q14 Days. In some embodiments, doxorubicin is administered at a dose of 40-60 mg/m² IV q21-28 Days. In some embodiments, doxorubicin is administered at a dose of 20 mg/m²/dose qweek.

In some embodiments, the additional therapeutic agent is epirubicin. In some embodiments, epirubicin is administered at a dose of 90 mg/m² IV on day 1, every 3 weeks.

In some embodiments, the additional therapeutic agent is etoposide. In some embodiments, etoposide is administered at a dose of 50-100 mg/m²/day IV on days 1-5. In some embodiments, etoposide is administered at a dose of 100 mg/m²/day IV on days 1, 3, 5.

In some embodiments, the additional therapeutic agent is gemcitabine. In some embodiments, gemcitabine is administered at a dose of 1000 mg/m² IV infusion over 30 min once weekly×7 weeks. In some embodiments, gemcitabine administration is followed by rest 1 week. In some embodiments, gemcitabine is administered at a dose of 1000 mg/m² IV once weekly for 3 weeks of each 28-day cycle.

In some embodiments, the additional therapeutic agent is ifosfamide. In some embodiments, ifosfamide is administered at a dose of 1.2 g/m²/day IV infusion over 30 minutes on days 1-5 q3-4 wk.

In some embodiments, the additional therapeutic agent is irinotecan. In some embodiments, irinotecan is administered at a dose of 250-350 mg/m² IV on day 1; every 21 d. In some embodiments, irinotecan is administered at a dose of 150-180 mg/m² IV on day 1; every 14 d. In some embodiments, irinotecan is administered at a dose of or 125 mg/m² IV on days 1 and 8; every 21 d.

In some embodiments, the additional therapeutic agent is irinotecan liposome. In some embodiments, irinotecan liposome is administered at a dose of 70 mg/m² IV infused over 90 min q2 wk.

In some embodiments, the additional therapeutic agent is leucovorin (folinic acid). In some embodiments, leucovorin is administered at a dose of 400 mg/m² IV on day 1.

In some embodiments, the additional therapeutic agent is mitomycin. In some embodiments, mitomycin is administered at a dose of 20 mg/m² IV q6-8 Weeks. In some embodiments, mitomycin is administered at a dose of 10 mg/m² as IV bolus days 1-29; not to exceed 20 mg/dose.

In some embodiments, the additional therapeutic agent is oxaliplatin. In some embodiments, oxaliplatin is administered at a dose of 85 mg/m² IV.

In some embodiments, the additional therapeutic agent is paclitaxel. In some embodiments, paclitaxel is administered at a dose of 135-250 mg/m² IV on day 1; every 21 d. In some embodiments, paclitaxel is administered at a dose of or 80 mg/m2 IV on day 1 weekly; every 28 d.

In some embodiments, the additional therapeutic agent is trifluridine. In some embodiments, the additional therapeutic agents are trifluridine and tipiracil. In some embodiments, trifluridine and tipiracil are administered at a dose of 35 mg/m² PO BID on Days 1-5 and Days 8-12 of each 28-day cycle.

In some embodiments, the additional therapeutic agent is napabucasin. In some embodiments, napabucasin is administered orally as two daily doses of 240 mg.

In some embodiments, the additional therapeutic agent is bevacizumab. In some embodiments, bevacizumab is administered at a dose of 5-10 mg/kg IV q2 Weeks.

In some embodiments, the additional therapeutic agent is ramucirumab. In some embodiments, ramucirumab is administered at a dose of 8 mg/kg IV q2 wk; infuse over 1 hr.

In some embodiments, the additional therapeutic agent is ziv-aflibercept. In some embodiments, ziv-aflibercept is administered at a dose of 4 mg/kg IV infused over 1 hr q2 weeks.

In some embodiments, the additional therapeutic agent cetuximab. In some embodiments, cetuximab is administered at a dose of 400 mg/m² IV infuse over 2 hr; subsequent doses: 250 mg/m² IV infuse over 60 min qWeek.

In some embodiments, the additional therapeutic agent panitumumab. In some embodiments, panitumumab is administered at a dose of 6 mg/kg IV infusion over 60 minutes (over 90 minutes if dose>1 g) q14 Days.

In some embodiments, the additional therapeutic agent is erlotinib. In some embodiments, erlotinib is administered at a dose of 100-150 mg/day PO.

In some embodiments, the additional therapeutic agent is trastuzumab. In some embodiments, trastuzumab is administered at a dose of 8 mg/kg IV loading dose on day 1 of cycle one, then 6 mg/kg IV; every 21 d with chemotherapy or 6 mg/kg IV loading dose on day 1 of cycle one, then 4 mg/kg IV every 14 d with chemotherapy.

In some embodiments, the additional therapeutic agent is lapatinib. In some embodiments, lapatinib is administered at a dose of 1250-1500 mg PO qDay on Days 1-21.

In some embodiments, the additional therapeutic agent is pertuzumab. In some embodiments, pertuzumab is administered at a dose of 840 mg IV infusion over 60 min, then 420 mg IV infusion over 30-60 min q3 Weeks.

In some embodiments, the additional therapeutic agent is trastuzumab emtansine. In some embodiments, trastuzumab emtansine is administered at a dose of 3.6 mg/kg IV infusion q3 weeks.

In some embodiments, the additional therapeutic agent is regorafenib. In some embodiments, regorafenib is administered at a dose of 160 mg (four 40-mg tablets) PO qDay for the first 21 days of each 28-day cycle.

In some embodiments, the additional therapeutic agent is sorafenib. In some embodiments, sorafenib is administered at a dose of 400 mg PO q12 hr.

In some embodiments, the additional therapeutic agent is apatinib mesylate. In some embodiments, apatinib mesylate is administered at a dose of 500 mg orally once a day every 4 weeks.

In some embodiments, the additional therapeutic agent is a check-point inhibitor of the PD-1 receptor. In some embodiments, the additional therapeutic agent is a check-point inhibitor of CTLA-4. In some embodiments, the additional therapeutic agent is nivolumab. In some embodiments, nivolumab is administered at a dose of 240 mg IV q2 Weeks infused over 30 min. In some embodiments, the additional therapeutic agent is pembrolizumab. In some embodiments, pembrolizumab is administered at a dose of 200 mg IV q3 Weeks.

In some embodiments, the additional therapeutic agent is sulindac. In some embodiments, sulindac is administered at a dose of 150-200 mg PO q12 hr.

In some embodiments, the additional therapeutic agent is aspirin. In some embodiments, aspirin is administered at a dose of 100-300 mg PO q12 hr.

In some embodiments, the additional therapeutic agent is celecoxib. In some embodiments, celecoxib is administered at a dose of 100 mg Celebrex twice daily, or 400 mg Celebrex twice daily.

In some embodiments, the additional therapeutic agent is tretinoin. In some embodiments, tretinoin is administered at a dose of 45 mg/m²/day PO divided q12 hr.

In some embodiments, the additional therapeutic agent is DFMO. In some embodiments, DFMO is administered at a dose of 500 mg qDay.

In some embodiments, the additional therapeutic agent is Na butyrate. In some embodiments, Na butyrate is administered at a dose to be assessed in Phase 1 trials.

In some embodiments, the additional therapeutic agent is a survivin inhibitor. In some embodiments, the survivin inhibitor is administered at a dose of to be assessed in Phase 1 trials. In some embodiments, the additional therapeutic agent is AICAR. In some embodiments, the additional therapeutic agent is Arctigenin. In some embodiments, the additional therapeutic agent is Cephalochromin. In some embodiments, the additional therapeutic agent is FL118. In some embodiments, the additional therapeutic agent is Flavopiridol. In some embodiments, the additional therapeutic agent is ICG-001. In some embodiments, the additional therapeutic agent is KPT-185. In some embodiments, the additional therapeutic agent is Lapatinib. In some embodiments, the additional therapeutic agent is MK-2206. In some embodiments, the additional therapeutic agent is NU6140. In some embodiments, the additional therapeutic agent is Panepoxydone. In some embodiments, the additional therapeutic agent is Piperine. In some embodiments, the additional therapeutic agent is Purvalanol A. In some embodiments, the additional therapeutic agent is Shepherdin. In some embodiments, the additional therapeutic agent is Terameprocol. In some embodiments, the additional therapeutic agent is UC112. In some embodiments, the additional therapeutic agent is YM155.

In some embodiments, the additional therapeutic agent is cortistatin. In some embodiments, cortistatin is administered at a dose to be assessed in Phase 1 trials.

In some embodiments, the additional therapeutic agent is cyclo somatostatin. In some embodiments, cyclo somatostatin is administered at a dose to be assessed in Phase 1 trials.

In some embodiments, the additional therapeutic agent is sandostatin. In some embodiments, sandostatin LAR (octreotide) is administered at a dose of 100-600 mcg/day SC divided q6-12 hr.

In some embodiments, the additional therapeutic agent is glucagon. In some embodiments, glucagon is administered at a dose of 0.2-1 mg (1 unit) IM/SC/IV.

In some embodiments, the additional therapeutic agent is a glucagon-like peptide. In some embodiments, the glucagon-like peptide is administered at a dose to be assessed in Phase 1 trials. In some embodiments, the additional therapeutic agent is somatostatin. In some embodiments, the additional therapeutic agent is a glucagon analogue. In some embodiments, the additional therapeutic agent is cortistatin. In some embodiments, the additional therapeutic agent is cyclo somatostatin. In some embodiments, the additional therapeutic agent is sandostatin LAR (octreotide). In some embodiments, the additional therapeutic agent is glucagon.

In some embodiments, the additional therapeutic agent is antabuse. In some embodiments, antabuse is administered at a dose of 125-500 mg PO qDay.

In some embodiments, the additional therapeutic agent is a TCF-4 inhibitor. In some embodiments, the TCF-4 inhibitor is administered at a dose to be assessed in Phase 1 trials. In some embodiments, the additional therapeutic agent is an anti-RPL29 antibody. In some embodiments, the additional therapeutic agent is BHQ880. In some embodiments, the additional therapeutic agent is CGP04909. In some embodiments, the additional therapeutic agent is CWP232291. In some embodiments, the additional therapeutic agent is DKN-01. In some embodiments, the additional therapeutic agent is G007-LK. In some embodiments, the additional therapeutic agent is G244-LM. In some embodiments, the additional therapeutic agent is ICG-001. In some embodiments, the additional therapeutic agent is IWR-1. In some embodiments, the additional therapeutic agent is JW55. In some embodiments, the additional therapeutic agent is LGK974. In some embodiments, the additional therapeutic agent is OMP-18R5. In some embodiments, the additional therapeutic agent is OMP-54F28. In some embodiments, the additional therapeutic agent is PKF115-584. In some embodiments, the additional therapeutic agent is PRI-724. In some embodiments, the additional therapeutic agent is Rp-8-Br-cAMPS. In some embodiments, the additional therapeutic agent is XAV939.

In some embodiments, the additional therapeutic agent is a bone morphogenic protein. In some embodiments, the bone morphogenic protein is administered at a dose to be assessed in Phase 1 trials. In some embodiments, the additional therapeutic agent is a bone morphogenic protein selected from BMP1, BMP2, BMP3, BMP4, BMP5, BMP6, BMP7, BMP8a, BMP8b, BMP10, BMP11, and BMP15.

In some embodiments, the additional therapeutic agent is atorvastatin. In some embodiments, atorvastatin is administered at a dose of 10-80 mg PO qDay.

In some embodiments, the additional therapeutic agent is fluvastatin. In some embodiments, fluvastatin is administered at a dose of 20-80 mg PO qDay.

In some embodiments, the additional therapeutic agent is lovastatin. In some embodiments, lovastatin is administered at a dose of 10-80 mg PO qDay.

In some embodiments, the additional therapeutic agent is pravastatin. In some embodiments, pravastatin is administered at a dose of 10-80 mg PO qDay.

In some embodiments, the additional therapeutic agent is rosuvastatin. In some embodiments, rosuvastatin is administered at a dose of 5-40 mg PO qDay.

In some embodiments, the additional therapeutic agent is simvastatin. In some embodiments, simvastatin is administered at a dose of 5-40 mg PO qDay.

In some embodiments, the additional therapeutic agent is pitavastatin. In some embodiments, pitavastatin is administered at a dose of 2-4 mg PO qDa.

The present disclosure provides pharmaceutically-acceptable salts of any therapeutic agent and/or compound described herein. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some cases, a pharmaceutically-acceptable salt is a metal salt. In some cases, a pharmaceutically-acceptable salt is an ammonium salt.

Acid addition salts can arise from the addition of an acid to a compound described herein. In some cases, the acid is organic. In some cases, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound described herein. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrazole, imidazole, pyrazine, piperazine, ethylenediamine, N,N′-dibenzylethylene diamine, procaine, chloroprocaine, choline, dicyclohexyl amine, and N-methylglucamine.

Non-limiting examples of suitable ammonium salts include is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrazole salt, an imidazole salt, a pyrazine salt, a piperazine salt, an ethylene diamine salt, an N,N′-dibenzylethylene diamine salt, a procaine salt, a chloroprocaine salt, a choline salt, a dicyclohexyl amine salt, and a N-methylglucamine salt.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzene sulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

The compounds and therapeutic agents described herein may in some cases exist as diastereomers, enantiomers, or other stereoisomeric forms. The compounds and therapeutic agents presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The compounds and therapeutic agents described herein include the use of amorphous forms as well as crystalline forms (also known as polymorphs). The compounds and therapeutic agents described herein may be in the form of pharmaceutically acceptable salts. As well, active metabolites of these compounds and therapeutic agents having the same type of activity are included in the scope of the present disclosure. In addition, the compounds and therapeutic agents described herein can exist in unsolvated as well as solvated forms with pharmaceutically acceptable solvents such as water, ethanol, and the like. The solvated forms of the compounds and therapeutic agents presented herein are also considered to be disclosed herein.

The compounds and therapeutic agents described herein include compounds and therapeutic agents that exhibit their natural isotopic abundance, and compounds and therapeutic agents where one or more of the atoms are artificially enriched in a particular isotope having the same atomic number, but an atomic mass or mass number different from the atomic mass or mass number predominantly found in nature. All isotopic variations of the compounds and therapeutic agents of the present invention, whether radioactive or not, are encompassed within the scope of the present invention. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared.

Compounds and therapeutic agents described herein having carbon-carbon double bonds or carbon-nitrogen double bonds may exist, where applicable, in Z- or E-form (or cis- or trans-form). Furthermore, some chemical entities may exist in various tautomeric forms. Unless otherwise specified, chemical entities described herein are intended to include all Z-, E- and tautomeric forms as well.

In certain cases, a compound and therapeutic agent described herein may be a prodrug, e.g., wherein a carboxylic acid present in the parent compound is presented as an ester. The term “prodrug” is intended to encompass compounds which, under physiologic conditions, are converted into pharmaceutical agents, i.e., parent compound, of the present disclosure. One method for making a prodrug is to include one or more selected moieties which are hydrolyzed under physiologic conditions to reveal the desired molecule. In certain embodiments, the prodrug is converted by an enzymatic activity of the host animal such as enzymatic activity in specific target cells in the host animal. For example, esters or carbonates (e.g., esters or carbonates of alcohols or carboxylic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. For example, the prodrug may have improved cell permeability over the parent compound. The prodrug may also have improved solubility in pharmaceutical formulations over the parent drug. In some embodiments, the design of a prodrug increases the lipophilicity of the pharmaceutical agent. In some embodiments, the design of a prodrug increases the effective water solubility.

The disclosure provides methods of preventing, or reducing, a relapse of a cancer in a subject in need thereof. The disclosure also provides methods of preventing, or reducing, a relapse of FAP in a subject in need thereof. The disclosure also provides methods of preventing, or reducing, a relapse of an APC mutation related disease or disorder in a subject in need thereof. In certain embodiments, the term “prevent” or “preventing” as related to a disease or disorder may refer to a compound or combination that, in a statistical sample, reduces the occurrence of the disorder or condition in the treated sample relative to an untreated control sample, or delays the onset or reduces the severity of one or more symptoms of the disorder or condition relative to the untreated control sample. The method includes administering a combination therapy described herein to treat minimal residual disease, and/or as maintenance therapy, e.g., as a prolonged or extended therapy after cessation of another cancer treatment. For example, the combination therapy may be administered after cessation of another therapy, such as chemotherapy, radiation therapy, and/or surgery.

In some aspects, combinations described herein, e.g., combinations of budesonide and an additional therapeutic agent described herein can be utilized for the treatment of cancer, for example, but not limited to, colorectal cancer. A combination therapy described herein can reduce the likelihood of metastasis in a subject in need thereof. In some embodiments, the metastasis is a solid tumor. In some embodiments, the metastasis is a liquid tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein can also be used for inhibiting progression of metastatic cancer tumors. Non-limiting examples of cancers include adrenocortical carcinoma, childhood adrenocortical carcinoma, AIDS-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, AIDS-related lymphoma, Burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

The combination therapies described herein may be used together with other therapies such as radiation therapy. Chemotherapy and radiotherapy treatment regimens can comprise a finite number of cycles of on-drug therapy followed by off-drug therapy, or comprise a finite timeframe in which the chemotherapy or radiotherapy is administered. The protocols can be determined by clinical trials, drug labels, and clinical staff in conjunction with the subject to be treated. The number of cycles of a chemotherapy or radiotherapy or the total length of time of a chemotherapy or radiotherapy regimen can vary depending on the subject's response to the cancer therapy. A pharmaceutical agent described herein can be administered after the treatment regimen of chemotherapy or radiotherapy has been completed.

In some aspects, the combinations described herein can be utilized to treat a subject in need thereof. In some cases, the subject to be treated by methods and compositions disclosed herein can be a human subject. A subject to be treated by methods and compositions disclosed herein can be a non-human animal. Non-limiting examples of non-human animals can include a non-human primate, a livestock animal, a domestic pet, and a laboratory animal.

In certain embodiments, the combination therapies described herein may be administered as separate agents or may be combined into a single pharmaceutical composition. For example, a combination of budesonide and an additional therapeutic agent described herein may be formulated as two separate pharmaceutical compositions, or may be co-formulated as a single pharmaceutical composition.

In certain embodiments, the disclosure provides a pharmaceutical composition, e.g., for oral or parenteral administration, comprising budesonide or a salt thereof. In some aspects, the pharmaceutical composition comprises budesonide or a salt thereof in an amount of at least about 1 mg to about 1000 mg, from about 100 mg to about 400 mg, from about 100 mg to about 200 mg, from about 200 mg to about 400 mg, or from about 250 mg to about 350 mg. For example, a pharmaceutical composition of the disclosure may comprise about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg of budesonide or a salt thereof. For a compound described herein, e.g., budesonide, formulated into a pharmaceutical composition in the form of a salt, the amount of the compound may reflect the free base weight and not the weight of the salt form. In certain embodiments, the pharmaceutical composition of budesonide or a salt thereof, does not include an additional anticancer agent. In certain embodiments, the pharmaceutical composition includes an additional anticancer agent as described herein.

A therapeutically effective amount of budesonide or a salt thereof can be expressed as mg of the compound per kg of subject body mass. In some instances, a dose of a therapeutically effective amount may be at least about 0.1 mg/kg to about 20 mg/kg, for example, about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 20 mg/kg. For a compound described herein, e.g., budesonide, formulated into a pharmaceutical composition in the form of a salt, the therapeutically effective amount of the compound may reflect the free base weight and not the weight of the salt form.

In certain embodiments, the disclosure provides a pharmaceutical composition, e.g., for oral or parenteral administration, comprising budesonide or a salt thereof. The pharmaceutical composition may comprise budesonide or a salt thereof in an amount of at least about 1 mg to about 1000 mg, from about 100 mg to about 1000 mg, from about 100 mg to about 800 mg, from about 200 mg to about 800 mg, or from about 300 mg to about 8000 mg. For example, a pharmaceutical composition of the disclosure may comprise about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, or about 1000 mg of budesonide or a salt thereof.

In certain embodiments, the disclosure provides a pharmaceutical composition, e.g., for oral or parenteral administration, comprising budesonide or a salt thereof. The pharmaceutical composition may comprise budesonide or a salt thereof in an amount of at least about 0.5 mg to about 50 mg, from about 1 mg to about 30 mg, from about 1 mg to about 20 mg, from about 1 mg to about 10 mg, or from about 1 mg to about 5 mg. For example, a pharmaceutical composition of the disclosure may comprise about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg of budesonide or a salt thereof.

In certain embodiments, formulations of the disclosure comprise budesonide or a salt thereof, wherein the budesonide or the salt thereof is about 70% to about 99.99%, about 80% to about 99.9%, about 85% to about 99%, about 90% to about 99%, about 95% to about 99%, about 97% to about 99%, about 98% to about 99%, about 98% to about 99.9%, about 99% to about 99.99%, about 99.5% to about 99.99%, about 99.6% to about 99.99%, about 99.8 to about 99.99%, or about 99.9% to about 99.99% free of impurities.

In certain embodiments, a pharmaceutical composition of the disclosure comprises both budesonide or a salt thereof and an additional therapeutic agent in amounts such as the ones described herein, e.g., a pharmaceutical composition with 100 to 400 mg of a budesonide or a salt thereof and 200 to 800 mg of an additional therapeutic agent. In certain embodiments, a pharmaceutical composition of the disclosure comprises both budesonide or a salt thereof, and an additional therapeutic agent, e.g., a pharmaceutical composition with 100 to 400 mg of budesonide or a salt thereof, and 1 to 10 mg of an additional therapeutic agent.

Pharmaceutical compositions disclosed herein may be in the form of a liquid formulation, a solid formulation or a combination thereof. Non-limiting examples of formulations may include a tablet, a capsule, a pill, a gel, a paste, a liquid solution and a cream. In some instances, budesonide or a salt thereof may be in a crystallized form. In pharmaceutical compositions comprising two or more therapeutic agents, each agent may be crystallized separately and then combined or they may be crystallized together. Compositions may comprise two or more therapeutic agents in one or more physical state. For example, a composition may be a tablet comprising one therapeutic agent in a solid formulation and another therapeutic agent or drug in a gel formulation. In certain embodiments, the composition is a single pharmaceutical composition comprising budesonide or a salt thereof in a first physical state and an additional therapeutic agent in a second physical state.

The compositions of the present disclosure may further comprise an excipient or an additive. Excipients may include any and all solvents, coatings, chelating agents, flavorings, colorings, lubricants, disintegrants, preservatives, sweeteners, anti-foaming agents, buffering agents, polymers, antioxidants, binders, diluents, and vehicles (or carriers). Generally, the excipient is compatible with the therapeutic compositions of the present disclosure.

Liquid preparations for oral administration can take the form of, for example, solutions, syrups or suspensions, or they can be presented as a dry product for reconstitution with water or other suitable vehicles before use. Such liquid preparations can be prepared by conventional approaches with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

This disclosure further encompasses anhydrous compositions and dosage forms comprising an active ingredient, since water can facilitate the degradation of some compounds. Anhydrous compositions and dosage forms of the present disclosure can be prepared using anhydrous or low moisture containing ingredients and low moisture or low humidity conditions. Compositions and dosage forms of the present disclosure which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials that prevent exposure to water such that they can be included in suitable formulary kits. Examples of suitable packaging include, but are not limited to, hermetically sealed foils, plastic, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

When aqueous suspensions and/or elixirs are desired for oral administration, the active ingredient therein can be combined with various sweetening or flavoring agents, coloring matter or dyes and, if so desired, emulsifying and/or suspending agents, together with such diluents as water, ethanol, propylene glycol, glycerin and various combinations thereof.

In one embodiment, the composition can include a solubilizer to ensure good solubilization and/or dissolution of the compound of the present disclosure and to minimize precipitation of the compound of the present disclosure. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

Pharmaceutical compositions described herein may be suitable for oral administration to a subject in need thereof. In some cases, slow release formulations for oral administration may be prepared in order to achieve a controlled release of the active agent in contact with the body fluids in the gastrointestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

In some embodiments, the compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. In some embodiments, the compositions can be formulated in a food composition. For example, the compositions can comprise a beverage, solid food, semi-solid food, or a food carrier.

In certain embodiments, the pharmaceutical formulations can be in a form suitable for parenteral injection as a sterile suspension, solution, or emulsion in oily or aqueous vehicles, and can contain formulation agents such as suspending, stabilizing, and/or dispersing agents. Pharmaceutical formulations for parenteral administration include, for example, aqueous solutions of the active compounds in water-soluble form. Suspensions of the active compounds can be prepared, for example, as oily injection suspensions. Suitable lipophilic solvents or vehicles include fatty oils such as sesame oil, or synthetic fatty acid esters, such as ethyl oleate, isopropyl palmitate, or medium chain triglycerides, or liposomes. In preferred embodiments, a formulation for parenteral administration is an aqueous suspension.

The therapeutic agents described herein may be present in a composition within a range of concentrations, the range being defined by an upper and lower value selected from any of the preceding concentrations. For example, the therapeutic agents of the disclosure may be present in the formulation at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

Methods for the preparation of compositions comprising the therapeutic agents described herein can include formulating the therapeutic agents with one or more inert, pharmaceutically-acceptable excipients. Liquid compositions include, for example, solutions in which one or more therapeutic agents are dissolved, emulsions comprising one or more therapeutic agents, or a solution containing liposomes, micelles, or nanoparticles comprising one or more therapeutic agents as disclosed herein. These compositions can also contain minor amounts of nontoxic, auxiliary substances, such as wetting or emulsifying agents, pH buffering agents, and other pharmaceutically-acceptable additives.

Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The compositions can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Pharmaceutical formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes.

A composition described herein, e.g., a pharmaceutical composition of budesonide or a salt thereof, or a co-formulation of budesonide or a salt thereof with an additional therapeutic agent, can be administered once or more than once each day. The composition may be administered serially (e.g., taken every day without a break for the duration of the treatment regimen). In some cases, the treatment regime can be less than a week, a week, two weeks, three weeks, a month, or greater than a month. In some cases, a composition of the disclosure is administered over a period of at least 12 weeks. In other cases, the composition is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In some cases, a therapeutically effective amount can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount can be administered one time per day, two times per day, three times per day, four times per day, five times per day, six times per day, seven times per day, eight times per day, nine times per day, 10 times per day, or greater than 10 times per day. In some cases, the composition is administered at least twice a day. In further cases, the composition is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day.

Pharmaceutical compositions of the disclosure can be administered either acutely or chronically. Pharmaceutical compositions of the invention can be administered as a single treatment or as a course of treatment. Treatments can be administered once per day, twice per day, three times per day, in the morning, in the evening, before sleeping, or continuously throughout the day. Treatments can be applied every day, every other day, every three days, twice weekly, once weekly, every other week, monthly, every six weeks, every other month, every three months, every six months, annually, every other year, every 5 years, or as required.

In certain embodiments, the dose of drug being administered may be temporarily reduced or temporarily suspended for a certain length of time. In certain embodiments, the patient will have a drug holiday wherein the patient does not receive the drug or receives a reduced amount of the drug for a period of time. A drug holiday can be, for example, between 2 days and 1 year, including by way of example only, 2 days, 3 days, 4 days, 5 days, 6 days, 7 days, 10 days, 12 days, 15 days, 20 days, 28 days, or more than 28 days. A drug holiday may be for about 1 month, about 2 months, about 3 months, about 4 months, about 5 months, about 6 months, about 7 months, about 8 months, about 9 months, about 10 months, about 11 months or about 12 months. The dose reduction during a drug holiday can be, for example, by 10%-100% of the original administered dose, including by way of example only 10%, 15%, 20%, 25%, 30%, 35%, 40%, 45%, 50%, 55%, 60%, 65%, 70%, 75%, 80%, 85%, 90%, 95%, and 100%. For further examples the dose reduction can be between 10% and 100%, between 20% and 80%, between 30% and 70%, between 50% and 90%, between 80% and 100% or between 90% and 100%.

Once improvement of the patient's conditions has occurred, a maintenance dose can be administered if necessary. Subsequently, the dosage or the frequency of administration, or both, can be reduced, as a function of the symptoms, to a level at which the improved disease, disorder or condition can be retained.

Additional methods for administering the combinations and formulations described herein include, for example, limited to delivery via enteral routes including oral, gastric or duodenal feeding tube, rectal suppository, rectal enema, parenteral routes, injection, infusion, intraarterial, intracardiac, intradermal, intraduodenal, intramedullary, intramuscular, intraosseous, intraperitoneal, intrathecal, intravascular, intravenous, intravitreal, intracameral, epidural, subcutaneous, inhalational, transdermal, transmucosal, sublingual, buccal, topical, epicutaneous, dermal, enema, ear drops, intranasal, and vaginal administration. The therapeutic agents described herein can be administered locally to the area in need of treatment, by for example, local infusion during surgery, topical application such as creams or ointments, injection, catheter, or implant. The administration can also be by direct injection at the site of a diseased tissue or organ.

The length of the period of administration and/or the dosing amounts can be determined by a physician or any other type of clinician. The physician or clinician can observe the subject's response to the administered compositions and adjust the dosing based on the subject's performance. For example, dosing for subjects that show reduced effects in energy regulation can be increased to achieve desired results.

In some embodiments, the combination therapies described herein can be administered together at the same time in the same route, or administered separately. In some embodiments, the components in the compositions can be administered using the same or different administration routes.

In some embodiment, the disclosure also provides for methods of manufacturing the compositions described herein. In some embodiments, the manufacture of a composition described herein comprises mixing or combining two or more components.

In some embodiments, the compositions can be combined or mixed with a pharmaceutically active or therapeutic agent, a carrier, and/or an excipient. Examples of such components are described herein. The combined compositions can be formed into a unit dosage as tablets, capsules, gel capsules, slow-release tablets, or the like.

In some embodiments, the composition is prepared such that a solid composition containing a substantially homogeneous mixture of the one or more components is achieved, such that the one or more components are dispersed evenly throughout the composition so that the composition can be readily subdivided into equally effective unit dosage forms such as tablets, pills and capsules.

A unit dose may be packaged into a container to be transferred to the user. A unit dose may be packaged in a tube, a jar, a box, a vial, a bag, a tray, a drum, a bottle, a syringe, or a can.

Another aspect of the disclosure provides for achieving desired effects in one or more subjects after administration of a combination composition described herein for a specified time period. For example, the beneficial effects of the compositions described herein can be observed after administration of the compositions to the subject for 1, 2, 3, 4, 6, 8, 10, 12, 24, or 52 weeks.

In certain embodiments, the combination therapies described herein may be administered by a combination treatment regimen. A combination treatment regimen can encompass treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. The disclosure also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

In certain embodiments, the combination therapy can provide a therapeutic advantage in view of the differential toxicity associated with the two treatment modalities. For example, treatment with budesonide can lead to a particular toxicity that is not seen with an additional therapeutic agent, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are synergistic, the doses of each of the agents can be reduced even further, thus lowering the associated toxicities to an even greater extent.

The therapeutic agents described herein or the pharmaceutically acceptable salts thereof, as well as combination therapies, may be administered before, during, or after the occurrence of a disease or condition, and the timing of administering the therapeutic agents, combination thereof, or composition containing therapeutic agents varies. The therapeutic agents described herein can be used as a prophylactic and may be administered continuously to subjects with a propensity to develop conditions or diseases in order to prevent the occurrence of the disease or condition. The compounds described herein and compositions thereof may be administered to a subject during or as soon as possible after the onset of the symptoms. A therapeutic agent described herein may be administered as soon as is practicable after the onset of a disease or condition is detected or suspected, and for a length of time necessary for the treatment of the disease.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Budesonide Prevents Intestinal Tumor Development Caused by Apc Mutations

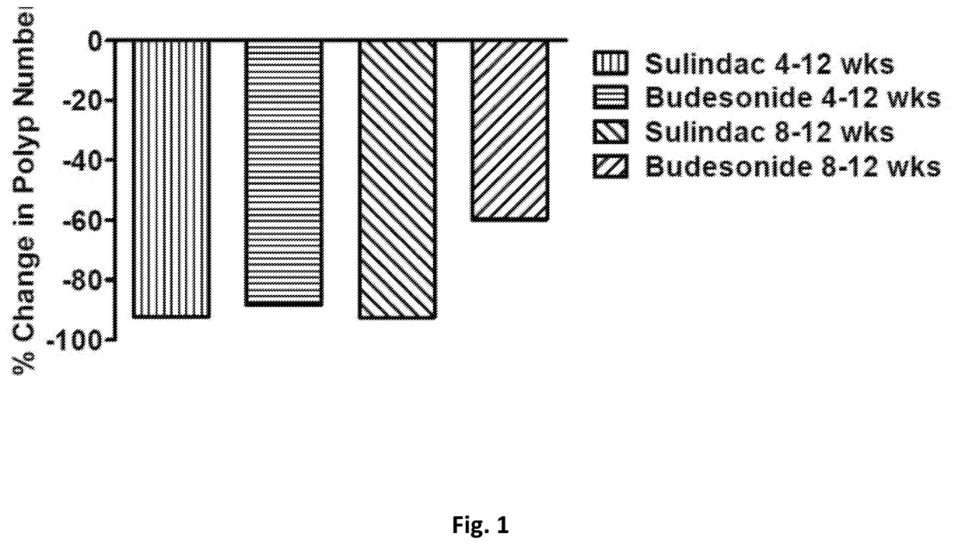

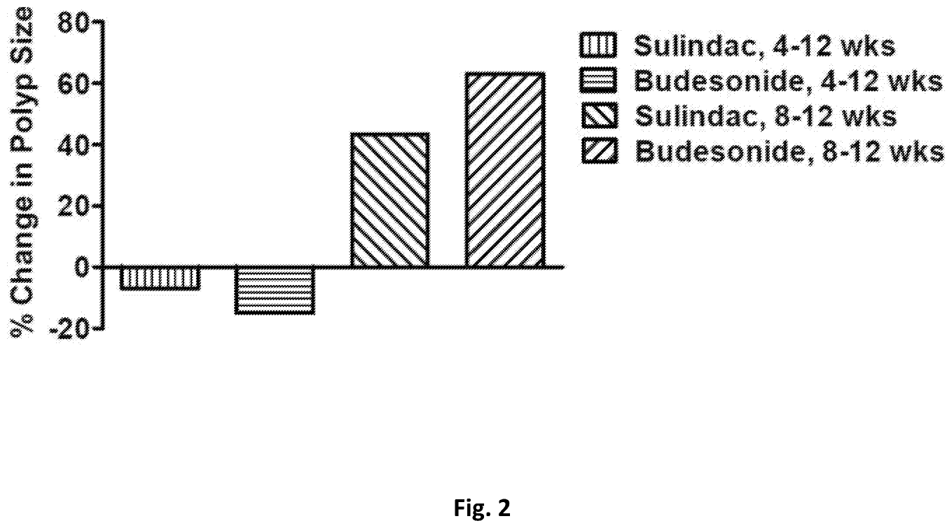

Budesonide, a steroidal anti-inflammatory drug active against lung cancer development, was investigated as a drug for CRC. Budesonide was evaluated using the murine model for hereditary CRC (FAP) in humans—Apc^{Min/+} mice—which, like FAP patients, carry a germline Apc mutation and which spontaneously develop multiple intestinal tumors. The effects of Budesonide were compared to Sulindac as the “gold standard” for an NSAID drug known to inhibit tumor development in Apc^{Min} mice. The Apc^{Min/+} and control mice (C57BL6) were given Budesonide- or Sulindac-containing diets starting at 4 or 8 weeks of age (corresponding, respectively, to ages before and after intestinal tumor development). At 12 weeks, mice were sacrificed and intestines analyzed for adenomas. Budesonide decreased tumor number by 88% (P=0.009) when started prior to the establishment of intestinal adenomas, and by 60% (p=0.03) when started after adenoma development (FIG. 1 illustrates the decrease in the number of intestinal tumors in Apc^{Min/+} mice given Budesonide- or Sulindac-containing diets for 8 or 4 weeks (from age 4 weeks to age 12 weeks or age 8 weeks to age 12 weeks); bars show % change in tumor number compared to untreated C57BL/6 control mice; each diet group had a total of 12 mice, six starting treatment at 4 weeks of age (before tumor initiation) and six starting at 8 weeks of age (after tumor initiation). Tumor size decreased when Budesonide was started before, but not after adenoma development (FIG. 2 illustrates the change in size of intestinal tumors in Apc^{Min/+} mice that were administered Budesonide- or Sulindac containing diets for 8 or 4 weeks (from age 4 weeks to age 12 weeks or age 8 weeks to age 12 weeks); bars show % change in tumor size compared to untreated C57BL/6 control mice; tumor size decreased when Budesonide or Sulindac was started before, but not after adenoma development.). Anti-tumor responses to Budesonide were comparable to those induced by the NSAID Sulindac administered before and after intestinal tumor development (90% and 90% decrease, respectively; FIG. 1). Without wishing to be bound by any particular theory, it is concluded that Budesonide has strong activity against the development of multiple tumors that would otherwise be induced by a variety of Apc mutations (combinations of inherited 1st hit and acquired 2nd hits), and also regression of many tumors after they had already developed.

### Example 2

### Mechanism of Action—Sulindac

Without wishing to be bound by any particular theory, it is believed that Sulindac causes regression of adenomas in FAP patients and inhibits adenoma formation in Apc^{Min} by several possible mechanisms. One molecular mechanism appears to involve Sulindac's ability, via its sulfide metabolite, to inhibit the enzymatic activity of both COX-1 and COX-2 and thereby inhibit prostaglandin synthesis. However, the effects of another metabolite, the sulfone product, appear to be COX-independent.

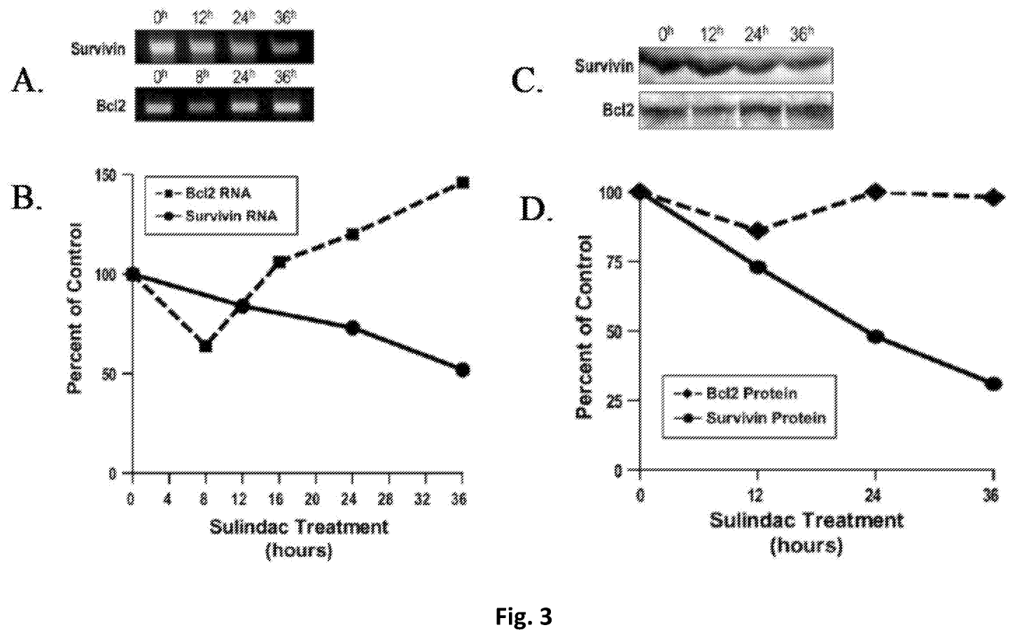

Another mechanism attributed to Sulindac's antitumor effects is its ability to induce apoptosis and decrease cell proliferation. This mechanism appears to involve a COX-independent mechanism whereby Sulindac (and other NSAIDs) induce degradation of beta-catenin, inactivation of Tcf-4, reduced expression of an anti-apoptotic protein (survivin; FIG. 3) and increased apoptosis in human CRC cells. FIGS. 3A-3D illustrate that Sulindac attenuates expression of the anti-apoptotic protein survivin in human CRC cells. To that end, survivin and Bcl-2 expression was evaluated in HT-29 cells that were treated with Sulindac (200 μM). RNA and protein expression were measured by RT-PCR and western blot, respectively. FIGS. 3A and 3B show an ethidium bromide-stained gel and graph of quantified data for survivin and Bcl-2 mRNA expression. FIGS. 3C and 3D show a western blot and graph of quantified data for survivin and Bcl-2 protein expression; Sulindac induces a monotonic decrease in survivin but not Bcl-2 expression in HT-29 cells. It also shows that Sulindac induces apoptosis in HT29 CRC cells by decreasing expression of Survivin (a WNT target gene).

### Example 3

### Mechanism of Action—Budesonide

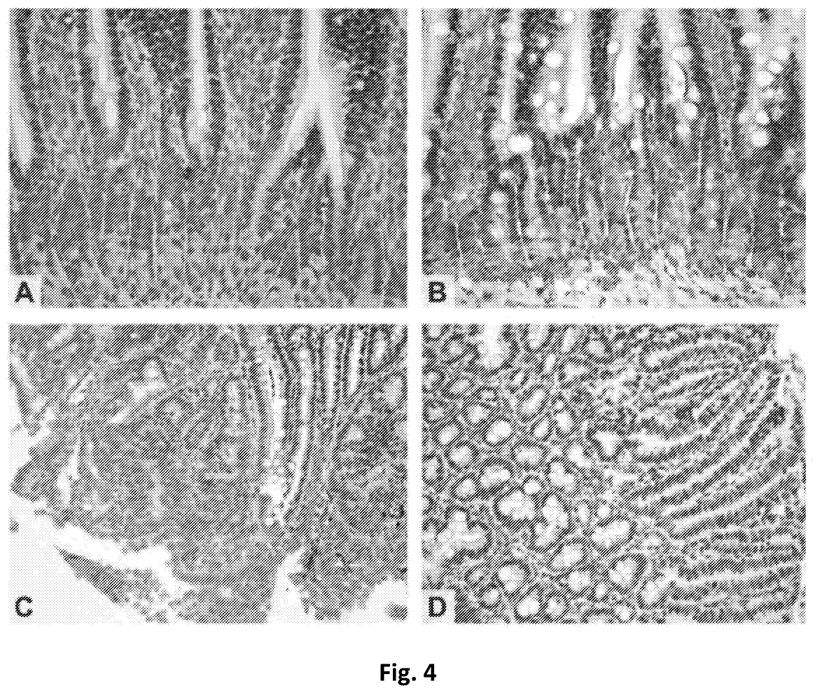

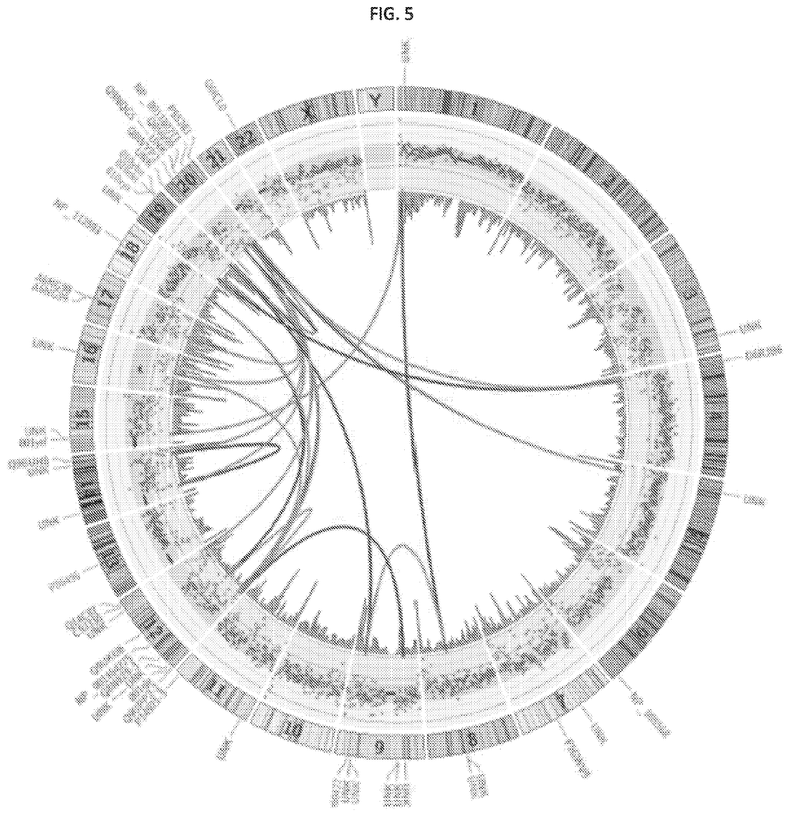

Without wishing to be bound by any particular theory, it is believed that Budesonide has a different mechanism of action than NSAIDs, including, but not limited to, by increasing DNA methylation of adenomas (FIG. 4) and human CRC cells (FIG. 5), which is consistent with reports that Budesonide induces re-methylation of lung DNA in association with its ability to prevent lung tumors in animal models. As shown in FIG. 4 regarding intestinal tumors from untreated Apc^{Min/+} mice, it appears that adenomas are hypo-methylated (FIG. 4), which is consistent with previous reports on DNA hypo-methylation being a hallmark of many tumors including CRC. This is also consistent with molecular studies wherein Apc^{Min/+} mice were crossed with mice that carry a germline mutation in the DNA methyltransferase (Dnmt1) gene, which leads to reductions in global DNA methylation. Such crossbred mice displayed an increased initiation of adenomas—tumors that also showed loss of the remaining wild-type Apc allele through loss of heterozygosity. In treated mice, it was found that Budesonide increased both the signal intensity and the proportion of adenoma cells staining positive for DNA methylation (FIG. 4). Thus, the current finding that Budesonide increases DNA methylation of adenomas corroborates the findings of Budesonide inducing DNA re-methylation in lung cancers and, without wishing to be bound by any particular theory, suggests one possible explanation for Budesonide's inhibition of intestinal tumor development.

FIGS. 4A-4D illustrate immunostaining using anti-5-methylcytidine antibodies to evaluate global DNA methylation; Budesonide increases global DNA methylation of intestinal tissues in Apc^{Min/+} Mice. Immunostaining signal intensity is decreased in the lower crypt region compared to the upper crypt and villous regions in normal-appearing small intestine of B6 controls (FIG. 4A) and Apc^{Min/+} mice (FIG. 4B). Immunostaining signal intensity is decreased in adenomas of the small intestine of Apc^{Min/+} mice (FIG. 4C). Budesonide treatment induces an increase in both the proportion and intensity of adenomatous cells staining positive for 5-methylcytidine (FIG. 4D). Budesonide increased both signal intensity (1.9 fold) and the proportion of cells staining positive for 5-methylcytidine in these adenomas.

FIG. 5 illustrates that Budesonide increases DNA methylation in human CRC cells. To determine if Budesonide increases DNA methylation in human CRC cells, epigenetic profiling was done on HT29 (APC mutant) cells in response to Budesonide exposure. The DNA methylation profiling revealed that an overall increase in methylation occurred across a large portion of the genome following Budesonide exposure. Indeed, 69.3% of 1 MB domains across the genome showed an increase in methylation in response to Budesonide versus control (FIG. 5). A statistically significant shift in CpG methylation occurred at 235 unique CpG sites (out of 1.9 M that were analyzed). The Figure shows a circular ideogram of methylation load with hypermethylated genes (>2-fold increase compared to mean) designated in red text and hypo-methylated genes (>2-fold decrease) in green text.

### Example 4

### Budesonide Preferentially Inhibits Growth of Malignant Human Colonic Cells that Carry an APC Mutation

Without wishing to be bound by any particular theory, it is believed that budesonide preferentially inhibits growth of malignant human colonic cells that carry an APC mutation. Also without wishing to be bound by any particular theory, it is believed that this effect is enhanced when Budesonide is combined with an additional therapeutic agent, for example, but not limited to, an NSAID. The effects of various therapeutic agents on CRC cells with mutant or wild type APC are analyzed in vitro. The ability of Budesonide to inhibit the in vitro growth of a variety of human CRC cell lines is confirmed. Budesonide selectively inhibits growth of cells with mutant APC relative to cells with wildtype APC. Budesonide inhibits intestinal tumor development and growth in Apc^{Min} mice that carry a germline mutation in the Apc tumor suppressor protein. Also, APC inactivation is a driver of human CRC growth. Without wishing to be bound by any particular theory, it is believed that drugs that inhibit intestinal tumor development in Apc^{Min} mice are candidate inhibitors of human CRC growth.

The antitumor activity of Budesonide against human CRC cells with mutant APC and with wildtype APC is evaluated. Five CRC cell lines with homozygous mutant APC (SW480, HT29, COLO320, CaCo2, DiFi) and two CRC cells lines with wild type APC (LoVo and HCT116) are evaluated. The anti-tumor effect of budesonide is evaluated by cell growth assays (counting number of cells over time, immunostaining for MCM2 and Ki67, MTT assay and BrdU uptake). Secondary outcome measures include: differentiation markers (CK20=pan-differentiation; Lectin=Paneth cells; Chromogranin A=enteroendocrine cells; intestinal alkaline phosphatase=absorptive cells; Muc2=Goblet cells), and apoptosis (survivin and Annexin V). Responses to drugs are determined by dose response and time course experiments. In time course experiments treated cells are sampled at 4 h and 8 h and then at 12 h intervals up to 48 h. In dose response experiments, budesonide is given in a dose range of 1-25 μM. All experiments are repeated at least three times on different days and with different cultures. Without wishing to be bound by any particular theory, it is believed that acceptance criteria are: cells with mutant APC are two fold (or more) as sensitive as wildtype APC cells to growth inhibition by budesonide. Increased differentiation and apoptosis occurs in parallel with growth inhibition.

Without wishing to be bound by any particular theory, it is believed that budesonide preferentially inhibits growth of CRC cells having mutant APC relative to cells with wildtype APC. Also without wishing to be bound by any particular theory, it is believed that cells with mutant APC are more sensitive to the growth inhibitory effects of Budesonide compared to wildtype APC cells. In some embodiments, the increased sensitivity is two-fold, or more. Without wishing to be bound by any particular theory, it is believed, that budesonide selectively targets APC mutant CRC cells. In some embodiments, mutant APC and wild type APC CRC cells are both growth inhibited (in some embodiments, by >50).

### Example 5

### NSAIDs Sulindac or Aspirin Potentiate the Anti-Tumor Activity of Budesonide

Without wishing to be bound by any particular theory, it is believed that budesonide in combination with other therapeutic agents, for example, but not limited to, NSAIDs, has synergistic anti-CRC effects. Also without wishing to be bound by any particular theory, it is believed that because both budesonide and sulindac inhibit tumor development and growth in Min mice and act via different mechanisms, budesonide synergizes with sulindac, or other NSAIDs, against CRC cells. When combined with another therapeutic agent, for example, but not limited to, an NSAID such as sulindac or aspirin, budesonide increases anti-CRC effects compared to administering either agent alone. A dose response analysis is done for sulindac (1-200 μM) and aspirin (0.1-10 mM) for five CRC cell lines with homozygous mutant APC (SW480, HT29, COLO320, CaCo2, DiFi) and two CRC cells lines with wild type APC (LoVo and HCT116). Combinations of an additional therapeutic agent, for example, but not limited to, an NSAID, and budesonide, at EC20 doses, are tested for synergistic activity. Higher doses (e.g., EC50 doses), or different treatment or time schedules, are also tried. For statistical analysis, data is expressed as mean±SEM (n=3). A student t-test is used to compare results between control and treated samples; when analyzing more than two groups ANOVA is used. Without wishing to be bound by any particular theory, it is believed that acceptance criteria are: budesonide and NSAIDs have synergistic effects.

Without wishing to be bound by any particular theory, it is believed that the effect of budesonide and an additional therapeutic agent administered together, for example, but not limited to, sulindac, is greater than the sum of their separate effects, consistent with a synergistic effect. In some embodiments, the effect of the agents in combination is equal to sum of the effect of budesonide and an additional therapeutic agent, for example, but not limited to, sulindac, given separately, indicating an additive combination. Without wishing to be bound by any particular theory, it is believed that NSAIDs are associated with GI and cardiovascular toxicities. Budesonide is a corticosteroid having minimal systemic toxicity because it has extensive first pass hepatic metabolism by cytochrome P-450 enzymes. Prednisone (a corticosteroid like Budesonide) is often used in combination treatment regimens (along with chemotherapy) which are usually curative for several types of leukemia, lymphoma and other cancers.

All publications, patents, and patent applications mentioned in this specification are herein incorporated by reference to the same extent as if each individual publication, patent, or patent application was specifically and individually indicated to be incorporated by reference.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. Cancer Facts & Statistics, American Cancer Society, https://www.cancer.org

2. International Agency for Research on Cancer, Cancer Fact Sheets, http://globocan.iarc.fr

3. Individual Drug Labels. US Food and Drug Administration. www.FDA.gov. Market and product forecasts: top 20 oncology therapy brands. Data monitor 2011.

4. Szabo L, https://khn.org/news/dozens-of-new-cancer-drugs-do-little-to-improve-survival-frustratingpatients. Feb. 9, 2017.

5. Ellis L M, Blanke C D, Roach N, Losing “Losing the battle with cancer” JAMA Oncol. 1:3-14, 2015.

6. Fojo T, Mailankody S, Lo A. Unintended consequences of expensive cancer therapeutics—the pursuit of marginal indications and a me-too mentality that stifles innovation and creativity: the John Conley Lecture. JAMA Otolaryngol Head Neck Surg. 140:1225-36, 2014.

7. Rupp T, Zuckerman D. Quality of Life, Overall survival, and costs of cancer drugs approved based on surrogate endpoints. JAMA Intern Med. 177:276-277, 2017.

8. Kumar H, Fojo T, Mailankody S. An appraisal of clinically meaningful outcomes guidelines for oncology clinical trials. JAMA Oncol. 2:1238-40, 2016.

9. https://www.statnews.com/2017/03/08/immunotherapy-cancer-breakthrough/

10. htips://www.statnews.com/2016/09/25/cancer-immunotherapy-caution/

11. https://www.cancer.gov/news-events/cancer-currents-blog/2014/who-responds-immunotherapy

12. Boland P M, Ma W W, Immunotherapy for Colorectal Cancer. Cancers 2017, 9, 50; doi:10.3390/cancers9050050

13. Pereira M A, Li Y, Gunning W T, Kramer P M, Al-Yaqoub F, Lubet R A, et al. Prevention of mouse lung tumors by budesonide and its modulation of biomarkers. Carcinogenesis. 23:1185-92, 2002.

14. Pereira M A, Tao L H, Wang W, Gunning W T, Lubet R. Chemoprevention: mouse and lung tumor bioassay and modulation of DNA methylation as a biomarker. Exp Lung Res. 31:145-63, 2005.

15. Wang Y, Wen W, Yi Y, Zhang Z, Lubet R A, You M. Preventive Effects of Bexarotene and Budesonide in a Genetically Engineered Mouse Model of Small Cell Lung Cancer. Cancer Prey Res. 2:1059-64, 2009.

16. Wattenberg L W, Estensen R D. Studies of chemopreventive effects of budesonide on benzo[a]pyreneinduced neoplasia of the lung of female A/J mice. Carcinogenesis 18: 2015-17, 1997.

17. Wattenberg L W, Wiedmann T S, Estensen R D, Zimmerman C L, Steele V E, Kelloff G J. Chemoprevention of pulmonary carcinogenesis by aerosolized budesonide in female A/J mice. Cancer Res 57:5489-92, 1997.

18. Fu H, Zhang J, Pan J, Zhang Q, Lu Y, Wen W, et al. Chemoprevention of lung carcinogenesis by the combination of aerosolized budesonide and oral pioglitazone in A/J mice. Mol Carcinog 50:913-21, 2011.

19. Rayyan Y, Williams J, Rigas B. The role of NSAIDs in the prevention of colon cancer. Cancer Invest. 20:1002-11, 2002.

20. Antonakopoulos N, Karamanolis D G. The role of NSAIDs in colon cancer prevention. Hepatogastroenterology. 54:1694-700, 2007.

21. Sansbury L B, Millikan R C, Schroeder J C, Moorman P G, North K E, Sandler R S. Use of nonsteroidal antiinflammatory drugs and risk of colon cancer in a population-based, case-control study of African Americans and Whites. Am J Epidemiol. 162:548-58, 2005.

22. Hua X, Phipps A I, Burnett-Hartman A N, Adams S V, Hardikar S, Cohen S A, Kocarnik J M, Ahnen D J, Lindor N M, Baron J A, Newcomb P A. Timing of aspirin and other nonsteroidal anti-inflammatory drug use among patients with colorectal cancer in relation to tumor markers and survival. J Clin Oncol. 35:2806-2813, 2017.

23. Walker A J, Grainge M J, Card T R. Aspirin and other non-steroidal anti-inflammatory drug use and colorectal cancer survival: A cohort study. Br J Cancer 107:1602-07, 2012.

24. Mccowan C, Munro A J, Donnan P T, Steele R J C. Use of aspirin post-diagnosis in a cohort of patients with colorectal cancer and its association with all-cause and colorectal cancer specific mortality. Eur J Cancer 49:1049-57, 2013.

25. Chan A T, Ogino S, Fuchs C S. Aspirin use and survival after diagnosis of colorectal cancer. JAMA 302:649-58, 2009.

26. Li P, Wu H, Zhang H, et al: Aspirin use after diagnosis but not prediagnosis improves established colorectal cancer survival: A meta-analysis. Gut 64:1419-1425, 2015.

1. A method of treating cancer, comprising administering to a subject in need thereof a therapeutically effective amount of budesonide, or a pharmaceutically acceptable salt, solvate, hydrate, cocrystal, or prodrug thereof.

2. The method of claim 1, wherein the cancer is selected from acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, and Waldenström's macroglobulinemia.

3. The method of claim 1, wherein the cancer is selected from colon cancer, stomach cancer, breast cancer, lung cancer, prostate cancer, pancreatic cancer, melanoma, and urothelial cancer.

4. The method of claim 1, wherein the cancer is colorectal cancer.

5. A method of treating familial adenomatous polyposis (FAP), comprising administering to a subject in need thereof a therapeutically effective amount of budesonide, or a pharmaceutically acceptable salt, solvate, hydrate, cocrystal, or prodrug thereof.

6. The method of any one of claims 1-5, further comprising administering to the subject a therapeutically effective amount of an additional therapeutic agent, or a pharmaceutically acceptable salt, solvate, hydrate, cocrystal, or prodrug thereof.

7. The method of claim 6, wherein the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil.

8. The method of claim 6, wherein the additional therapeutic agent is selected from bevacizumab, ramucirumab, and ziv-aflibercept.

9. The method of claim 6, wherein the additional therapeutic agent is selected from cetuximab, panitumumab, erlotinib, trastuzumab, lapatinib, pertuzumab, and trastuzumab emtansine.

10. The method of claim 6, wherein the additional therapeutic agent is selected from regorafenib, sorafenib, and apatinib.

11. The method of claim 6, wherein the additional therapeutic agent is a check-point inhibitor of the PD-1 receptor or the CTLA-4 receptor.

12. The method of claim 6, wherein the additional therapeutic agent is napabucasin.

13. The method of claim 6, wherein the additional therapeutic agent is selected from sulindac, aspirin, celecoxib, difluoromethylomithine (DFMO), sodium butyrate, cyclosomatostatin, somatostatin, glucagon and antabuse.

14. The method of claim 6, wherein the additional therapeutic agent is a glucagon-like peptide.

15. The method of claim 6, wherein the additional therapeutic agent is a statin.

16. The method of claim 6, wherein the additional therapeutic agent is an all-trans retinoid or a 9-cis retinoid.

17. The method of claim 6, wherein the additional therapeutic agent is a survivin inhibitor.

18. The method of claim 6, wherein the additional therapeutic agent is a TCF-4 inhibitor.

19. The method of claim 6, wherein the additional therapeutic agent is a bone morphogenic protein.

20. The method of any one of claims 6-19, wherein budesonide and the additional therapeutic agent are administered concurrently.

21. The method of any one of claims 6-19, wherein budesonide and the additional therapeutic agent are administered sequentially within about 1 hour to about 24 hours of each other.

22. The method of any one of claims 6-19, wherein budesonide and the additional therapeutic agent are administered sequentially within about 1 day to about 7 days of each other.

23. The method of any one of claims 6-20, wherein budesonide and the additional therapeutic agent are co-formulated in a pharmaceutical composition.

24. The method of any one of claims 6-23, wherein budesonide and the additional therapeutic agent are administered daily, every other day, or every third day.

25. A pharmaceutical composition comprising:
a therapeutically effective amount of budesonide, or a pharmaceutically acceptable salt, solvate, hydrate, cocrystal, or prodrug thereof;
an additional therapeutic agent, or a pharmaceutically acceptable salt, solvate, hydrate, cocrystal, or prodrug thereof; and
a pharmaceutically acceptable excipient.

26. The pharmaceutical composition of claim 25, wherein the additional therapeutic agent is selected from 5-fluorouracil, albumin-bound paclitaxel, capecitabine, carboplatin, cisplatin, dacarbazine, docetaxel, doxorubicin, epirubicin, etoposide, gemcitabine, ifosfamide, irinotecan, irinotecan liposome, leucovorin (folinic acid), mitomycin, oxaliplatin, paclitaxel, trifluridine, and tipiracil.

27. The pharmaceutical composition of claim 25, wherein the additional therapeutic agent is selected from bevacizumab, ramucirumab, and ziv-aflibercept.

28. The pharmaceutical composition of claim 25, wherein the additional therapeutic agent is selected from cetuximab, panitumumab, erlotinib, trastuzumab, lapatinib, pertuzumab, and trastuzumab emtansine.

29. The pharmaceutical composition of claim 25, wherein the additional therapeutic agent is selected from regorafenib, sorafenib, and apatinib.

30. The pharmaceutical composition of claim 25, wherein the additional therapeutic agent is a check-point inhibitor of the PD-1 receptor or the CTLA-4 receptor.

31. The pharmaceutical composition of claim 25, wherein the additional therapeutic agent is napabucasin.

32. The pharmaceutical composition of claim 25, wherein the additional therapeutic agent is selected from sulindac, aspirin, celecoxib, difluoromethylomithine (DFMO), sodium butyrate, cyclosomatostatin, somatostatin, glucagon and antabuse.

33. The pharmaceutical composition of claim 25, wherein the additional therapeutic agent is a glucagon-like peptide.

34. The pharmaceutical composition of claim 25, wherein the additional therapeutic agent is a statin.

35. The pharmaceutical composition of claim 25, wherein the additional therapeutic agent is an all-trans retinoid or a 9-cis retinoid.

36. The pharmaceutical composition of claim 25, wherein the additional therapeutic agent is a survivin inhibitor.

37. The pharmaceutical composition of claim 25, wherein the additional therapeutic agent is a TCF-4 inhibitor.

38. The pharmaceutical composition of claim 25, wherein the additional therapeutic agent is a bone morphogenic protein.
